(19) United States
(12) Patent Application Publication
(10) Pub. No.: US 2019/0351177 A1
(43) Pub. Date: Nov. 21, 2019

(54) MIXED GAS SUPPLYING APPARATUS

(71) Applicant: NIHON KOHDEN CORPORATION, Shinjuku-ku, Tokyo (JP)

(72) Inventors: Makoto HIGUCHI, Tokorozawa-shi, Saitama (JP); Kentaro SUZUKI, Kamiina-gun, Nagano (JP); Toshihiro URANO, Kamiina-gun, Nagano (JP)

(21) Appl. No.: 16/477,324

(22) PCT Filed: Jan. 10, 2018
(86) PCT No.: PCT/JP2018/000320

§ 371 Date: Jul. 11, 2019

(30) Foreign Application Priority Data
Jan. 17, 2017 (JP) 2017-006203

(51) Int. Cl. A61M 16/10; A61M 16/12; A61M 16/20; A61M 16/00

(52) U.S. Cl. CPC A61M 16/108; A61M 16/12; A61M 16/201; A61M 16/024; A61M 2205/3327; A61M 2202/02; A61M 2016/0033; A61M 2205/3673; A61M 2205/3372; A61M 16/0003

(57) ABSTRACT

A temperature adjuster (20) is configured to adjust temperature associated with a first gas supplying medium (10) for providing first gas to be mixed. A first flow rate controller (30) is configured to control a flow rate of the first gas. A second flow rate controller (40) is configured to control a flow rate of second gas to be mixed that is different from the first gas. A mixer (50) is configured to supply mixed gas in which the first gas supplied by the first flow rate controller (30) and the second gas supplied by the second flow rate controller (40) have been mixed.

### TECHNICAL FIELD

The present disclosure relates to an apparatus for supplying mixed gas mainly used for medical treatment.

### BACKGROUND ART

It is known that hydrogen gas (H2) has various treatment effects, such as an anti-inflammatory effect. With attention paid to this anti-inflammatory effect of hydrogen gas, hydrogen has been used for medical treatment and health promotion (see Patent Literature 1).

### CITATION LIST

### Patent Literature

PTL 1: Japanese Patent Publication No. 2015-167926A

### SUMMARY OF INVENTION

### Technical Problem

Patent Literature 1 mentions that hydrogen is taken out from a hydrogen storage alloy and the hydrogen is added to medical electrolyte liquid. However, Patent Literature 1 mentions that hydrogen is added to liquid but fails to disclose that hydrogen gas having the concentration suitable for inhalation is supplied.

In a medical site, it is expected that a patient is let to inhale hydrogen gas having the concentration suitable for the content of treatment via an inhalation mask or an artificial respirator. In such a medical site, it is desired the provision of an apparatus for controlling the concentration of mixed gas containing hydrogen gas or the like to the concentration suitable for treatment and for supplying the gas.

In a hydrogen storage alloy, it is known that, when the temperature of the hydrogen storage alloy is set constant, equilibrium is maintained among hydrogen pressure P, hydrogen composition C (hydrogen concentration), and temperature T (a pressure-composition diagram, generally referred to as “PCT diagram”). It is also known that, when the temperature of the hydrogen storage alloy lowers, the hydrogen pressure also lowers, and the amount of hydrogen to be released lowers as well.

Hence, the temperature of the hydrogen storage alloy is required to be adjusted properly to generate hydrogen gas having the concentration suitable for the content of treatment using the hydrogen storage alloy.

The present disclosure is to provide a mixed gas supplying apparatus for supplying mixed gas containing first gas (preferably hydrogen gas) at the concentration suitable for the content of treatment.

### Solution to Problem

According to one aspect of the present disclosure, there is provided a mixed gas supplying apparatus comprising: a temperature adjuster configured to adjust temperature associated with a first gas supplying medium for providing first gas to be mixed; a first flow rate controller configured to control a flow rate of the first gas; a second flow rate controller configured to control a flow rate of second gas to be mixed that is different from the first gas; and a mixer configured to supply mixed gas in which the first gas supplied by the first flow rate controller and the second gas supplied by the second flow rate controller have been mixed.

With the above configuration, pressure of the first gas provided from the first gas supplying medium is controlled by adjusting the temperature associated with the first gas supplying medium, and the flow rate of the first gas is controlled by the first flow rate controller. Accordingly, it is possible to supply the mixed medium containing the first gas with concentration suitable for the treatment to be performed.

Therefore, it is possible to provide a mixed gas supplying apparatus capable of supplying mixed gas containing first gas with concentration suitable for the treatment to be performed.

### BRIEF DESCRIPTION OF DRAWINGS

FIG. 1 is a functional block diagram illustrating a mixed gas supplying apparatus according to a first embodiment.

FIG. 2 is a functional block diagram illustrating a mixed gas supplying apparatus according to a second embodiment.

FIG. 3 is a functional block diagram illustrating a mixed gas supplying apparatus according to a third embodiment.

FIG. 4 is a functional block diagram illustrating a mixed gas supplying apparatus according to a fourth embodiment.

FIG. 5 is a functional block diagram illustrating a mixed gas supplying apparatus according to a fifth embodiment.

### DESCRIPTION OF EMBODIMENTS

Examples of the embodiment of a mixed gas supplying apparatus will be described below in detail with reference to the accompanying drawings.

### First Embodiment

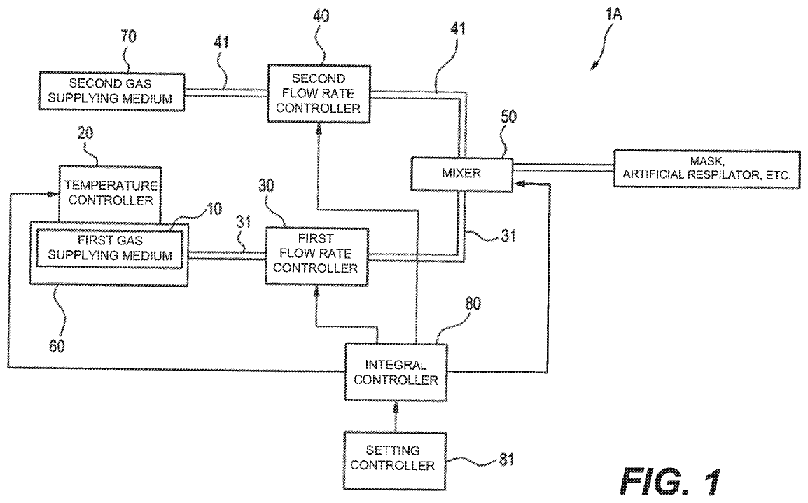

FIG. 1 is a functional block diagram illustrating a mixed gas supplying apparatus 1A according to a first embodiment. The mixed gas supplying apparatus 1A is an apparatus for providing first gas, such as hydrogen, from a first gas supplying medium 10 and for supplying mixed gas containing the first gas at the concentration suitable for treatment and health promotion.

As illustrated in FIG. 1, the mixed gas supplying apparatus 1A comprises a temperature adjuster 20, a first flow rate controller 30, a second flow rate controller 40, a mixer 50, and a holder 60. The first gas supplying medium 10 is configured to provide the first gas. The temperature adjuster 20 is configured to adjust the temperature associated with the first gas supplying medium 10. The first flow rate controller 30 is configured to control the flow rate of the first gas to be mixed. The second flow rate controller 40 is configured to control the flow rate of second gas to be mixed. The second gas is different from the first gas. The mixed gas supplying apparatus 1A is connected to a second gas supplying medium 70. The second gas is output from the second gas supplying medium 70 to the mixed gas supplying apparatus 1A. The mixer 50 is configured to mix the first gas output from the first flow rate controller 30 and the second gas output from the second flow rate controller 40. The mixer 50 is also configured to output the mixed gas. The first gas supplying medium 10 is held by the holder 60.

The mixed gas supplying apparatus 1A further comprises an integral controller 80 configured to integrally control the temperature adjuster 20, the first flow rate controller 30, the second flow rate controller 40, and the mixer 50 so that the first gas in the mixed gas has a desired concentration. The integral controller 80 is communicatively connected to the temperature adjuster 20, the first flow rate controller 30, the second flow rate controller 40 and the mixer 50. The processing performed by the integral controller 80 may be implemented when a CPU (not shown) disposed in the mixed gas supplying apparatus 1A executes software programs read out from a storage device such as a hard disk drive (not shown). At least a portion of the processing performed by the integral controller 80 may be implemented by at least one hardware such as electric circuits.

The first gas supplying medium 10, the first flow rate controller 30 and the mixer 50 are connected via a first gas supplying passage 31 composed of conveying members such as gas pipes. Furthermore, the second gas supplying medium 70, the second flow rate controller 40 and the mixer 50 are also connected via a second gas supplying passage 41 composed of conveying members such as gas pipes.

The first gas is gas contributing to the efficacy suitable for treatment and health promotion. In this embodiment, hydrogen having a treatment effect, such as anti-inflammation, is used as the first gas. The first gas supplying medium 10 is a medium configured to provide the first gas to the first flow rate controller 30. In this embodiment, a hydrogen storage alloy is used as the first gas supplying medium 10.

The first gas supplying medium 10 is held in the holder 60, such as a canister, in an airtight manner. The holder 60, for example, comprises a chamber (not shown) for accommodating the first gas supplying medium 10 therein and a supply valve (not shown) for providing the first gas to the first flow rate controller 30. The supply valve of the holder 60 is opened and closed manually or by the control of the integral controller 80 as described later.

The holder 60 can hold the first gas supplying medium 10 therein, and containers having various shapes and sizes can be used as the holder, provided that they can adjust the temperature of the first gas supplying medium 10 being held therein. The holder 60 may be equipped with a supply valve being opened/closed by control signals from the integral controller 80. The holder 60 may have a size being portable by hand. For example, as the holder 60, it is possible to use a small cylinder (not shown) incorporating the first gas supplying medium 10 in advance.

The temperature adjuster 20 is configured to adjust the temperature associated with the first gas supplying medium 10 for providing the first gas. The temperature associated with the first gas supplying medium 10 may be the temperature of the first gas, the temperature of the first gas supplying medium 10 or the temperature of the holder 60 for accommodating the first gas supplying medium 10 therein. For example, the temperature adjuster 20 may be an electric heater that is used to heat the holder 60 for holding the first gas supplying medium 10.

As the temperature associated with the first gas supplying medium 10 rises, the pressure of the hydrogen serving as the first gas inside the first gas supplying medium 10 also rises. The provision of the first gas to the first flow rate controller 30 is started by the rise of the pressure. The temperature adjuster 20 in the mixed gas supplying apparatus 1A adjusts the temperature associated with the first gas supplying medium 10.

The first flow rate controller 30 is configured to control the flow rate of the first gas to be mixed at the mixer 50. For example, the first flow rate controller 30 may be configured to have a mass flow controller provided with a supply valve (not shown) capable of controlling the amount of the first gas to be supplied to the mixer 50.

The second gas is gas that is different from the first gas and used to adjust the concentration of the first gas to such a value suitable for a person inhaling the mixed gas, such as a patient. The second gas is provided from the second gas supplying medium 70 to the second flow rate controller 40. In this embodiment, medical compressed air is used as the second gas. A medical compressed air supplying equipment provided in a hospital is used as the second gas supplying medium 70.

The second gas is not limited to the medical compressed air. For example, oxygen and the air in a room where home medical care is provided may be used as the second gas, provided that the second gas is gas different from the first gas and can be used to adjust the concentration of the first gas to such a value suitable for the inhalation.

The second flow rate controller 40 is configured to adjust the flow rate of the second gas to be mixed at the mixer 50. The second flow rate controller 40 may be, for example, a mass flow controller having a supply valve (not shown) disposed in the supplying passage 41 for supplying the second gas to the mixer 50. However, other various kinds of valves may also be provided.

The mixer 50 is configured to mix the first gas supplied from the first flow rate controller 30 and the second gas supplied from the second flow rate controller 40, thereby to prepare mixed gas in which the first gas is uniformly distributed at the desired concentration. The mixer 50 can be configured to have a space for holding the first gas and the second gas therein and to supply the mixed gas to a mask or the like. The mixer 50 may be equipped with a supply valve as necessary. The mixed gas prepared by the mixer 50 is supplied to the patient via the mask or the artificial respirator illustrated in FIG. 1.

The integral controller 80 may adjust the concentration of the first gas in the mixed gas of the mixed gas supplying apparatus 1A by a mass flow controller (not shown), or may control the concentration by using various kinds of target values and the actual measured values in the mixed gas supplying apparatus 1A.

The various kinds of target values to be stored in the integral controller 80 are, for example, the flow rate of the first gas to be supplied from the first flow rate controller 30 to the mixer 50 to be mixed; the amount of the second gas provided from the second gas supplying medium 70; the flow rate of the second gas used to be supplied from the second flow rate controller 40 to the mixer 50 to be mixed; and various kinds of relational expressions for setting the concentration of the first gas in the mixed gas prepared by the mixer 50 to a predetermined concentration value.

The target values to be stored in the integral controller 80 may be values determined by the programs registered in the integral controller 80 in advance or may be values input to the integral controller 80 from the outside and then registered. In the case that the target values are input to the integral controller 80 from the outside, the various kinds of target values may be registered by a setting controller 81 illustrated in FIG. 1. The setting controller 81 may be, for example, a button or a knob provided on the housing of the mixed gas supplying apparatus 1A or a remote controller for transmitting signals to the mixed gas supplying apparatus 1A.

Furthermore, the integral controller 80 is configured to be able to output instruction associated with the provision condition of the first gas and instruction associated with the provision condition of the second gas. The provision condition include a condition as to whether each gas can be provided, the flow rate, concentration, etc. of the gas to be provided, the timing for starting the provision of the gas to be provided, etc. In this embodiment, the integral controller 80 is configured to transmit an instruction for causing the second gas to be provided and an instruction for designating the flow rate of the second gas provided to the second flow rate controller 40. The integral controller 80 is also configured to transmit an instruction for causing the first gas to be provided and an instruction for designating the flow rate of the first gas to be provided to the first flow rate controller 30.

Moreover, the integral controller 80 may be configured to perform the initial setting of the temperature adjuster 20, the first flow rate controller 30 and the second flow rate controller 40 depending on the concentration of the first gas in the mixed gas having been set by the setting controller 81 (or a default value) at the time when the power source (not shown) of the mixed gas supplying apparatus 1A is turned on. More specifically, the integral controller 80 adjusts the temperature adjuster 20 depending on the temperature of the holder 60 immediately after the power source (not shown) of the mixed gas supplying apparatus 1A is turned on. Furthermore, after the adjustment of the temperature adjuster 20, the integral controller 80 performs control so that the flow rate of the first gas in the first flow rate controller 30 and the flow rate of the second gas in the second flow rate controller 40 are set to preset values. By virtue of the control of the integral controller 80, the concentration of the first gas in the mixed gas can be adjusted to the desired value only with an installing action of the holder 60 in the mixed gas supplying apparatus 1A.

Next, the operation for supplying the mixed gas using the mixed gas supplying apparatus 1A according to the first embodiment will be described with reference to FIG. 1.

The holder 60 should be installed in the mixed gas supplying apparatus 1A before the power source (not shown) is turned on. After that, the supply valve of the holder 60, the first flow rate controller 30 and the mixer 50 are communicated via the gas pipes serving as conveying members. As well, the supply valve of the second gas supplying medium 70, the second flow rate controller 40 and the mixer 50 are communicated via the gas pipes. At this time, the passage from the first flow rate controller 30 to the mixer 50 and the passage from the second flow rate controller 40 to the mixer 50 are still closed.

After the power source (not shown) of the mixed gas supplying apparatus 1A is turned on, an instruction for initiating the supply of a mixed gas is provided (for example, pressing of a start button). The integral controller 80 transmits instruction for causing the second gas supplying medium 70 to provide the second gas. Furthermore, the integral controller 80 controls the second flow rate controller 40 on the basis of the target values (default values may be used; information, such as the concentration of the first gas in the mixed gas and the flow rate of the mixed gas), thereby supplying the second gas having the desired flow rate to the mixer 50.

The integral controller 80 outputs an instruction as for the provision condition of the second gas to the second flow rate controller 40, thereby initiating the provision of the second gas from the second gas supplying medium 70 to the second flow rate controller 40. The integral controller 80 also transmits an instruction as for the flow rate of the second gas to be mixed in the second flow rate controller 40, thereby initiating the supply of the second gas from the second flow rate controller 40 to the mixer 50.

After the supply of the second gas to the mixer 50 is started, the integral controller 80 instructs the temperature adjuster 20 to raise the temperature. In response to this instruction, the temperature adjuster 20 raises the temperature of the holder 60. The integral controller 80 is configured to cause the first gas to be provided after the provision of the second gas is started. In other words, the integral controller 80 controls the first flow rate controller 30 and the second flow rate controller 40 so that the first gas is provided after the supply of the second gas to the mixer 50 is started. More specifically, the integral controller 80 causes the first gas supplying medium 10 to provide the first gas, transmits an instruction for designating the flow rate of the first gas to be mixed in the first flow rate controller 30, and causes the first flow rate controller 30 to supply the first gas to the mixer 50, after causing the second gas supplying medium 70 to provide the second gas, transmitting an instruction for designating the flow rate of the second gas to be mixed in the second flow rate controller 40, and causing the second flow rate controller 40 to supply the second gas to the mixer 50.

The mixer 50 mixes the first gas and the second gas, thereby preparing the mixed gas. The prepared mixed gas is inhaled by the patient via the mask or the artificial respirator illustrated in FIG. 1.

As described above, with the mixed gas supplying apparatus 1A according to this embodiment, the temperature adjuster 20 adjusts the temperature associated with the first gas supplying medium 10, and the first flow rate controller 30 adjusts the flow rate of the first gas to be mixed. Hence, the concentration of the mixed gas to be supplied from the mixer 50 to the patient can be adjusted to such a value suitable for treatment, whereby the mixed gas for inhalation having the concentration suitable for treatment can be supplied.

Conventionally, a 47 liter gas cylinder for providing hydrogen gas having a concentration of 1.3% for example is used to supply hydrogen gas for treatment. However, the concentration of the hydrogen cannot be adjusted, and the hydrogen gas thus cannot be supplied at the concentration suitable for the treatment to which the patient is to be subjected.

With the mixed gas supplying apparatus 1A according to this embodiment, since the flow rate of the first gas to be mixed can be controlled by the first flow rate controller 30, the concentration of the mixed gas can be adjusted to such a value suitable for the treatment to which the patient is to be subjected.

Furthermore, it is also conventionally known an apparatus for preparing hydrogen gas by mixing the hydrogen gas prepared by electrolyzing water with the air. However, the apparatus has a structure in which the water for use in the electrolysis remains inside the apparatus for a long time. Hence, there is a risk that germs are prepared and propagate inside the apparatus. Moreover, there is a risk that the electrodes for use in the electrolysis are stained with impurities attached thereto, for example.

With the mixed gas supplying apparatus 1A according to this embodiment, since the first gas can be directly taken out from the first gas supplying medium 10, such as a hydrogen storage alloy, such a risk of the stains and the propagation of germs as in the electrolysis type apparatus is avoided.

Furthermore, the integral controller 80 controls the first flow rate controller 30 and the second flow rate controller 40 such that the provision of the first gas is initiated after the provision of the second gas is initiated. Hence, the concentration of the first gas in the mixed gas rises gradually to the desired value, whereby the concentration can be controlled securely.

Since the first gas supplying medium 10 is held in the holder 60 in an air tight manner, the first gas supplying medium 10 can be conveniently carried and stored. Furthermore, in the case that the first gas provided from the first gas supplying medium 10 is used up completely, the holder 60 can be replenished with a holder 60 holding a new first gas supplying medium 10, whereby the mixed gas can be supplied continuously. Moreover, in the case that a compact gas cylinder is used as the holder 60, the holder 60 can be carried and stored more conveniently.

As mentioned above, Patent Literature 1 is a document describing that hydrogen is added to liquid. It is herein known that the saturation amount of hydrogen in liquid is determined at the same temperature and the same pressure. Hence, the supply of hydrogen to liquid in Patent Literature 1 does not require strict control.

However, in the case that gases are mixed directly, control stricter than that in the case that hydrogen is added to liquid is required. In other words, since the concentration of the first gas in the mixed gas prepared by the mixer 50 changes depending on the fluctuation of the temperature associated with the first gas supplying medium 10 and the concentration also changes depending on the increase/decrease of the provision amount of the first gas and the increase/decrease of the provision amount of the second gas even at the same temperature, various kinds of control are required.

Since the mixed gas supplying apparatus 1A according to this embodiment has the temperature adjuster 20 and the first flow rate controller 30, the concentration of the first gas in the mixed gas can be adjusted to such a value suitable for the treatment to which the patient is to be subjected.

### Second Embodiment

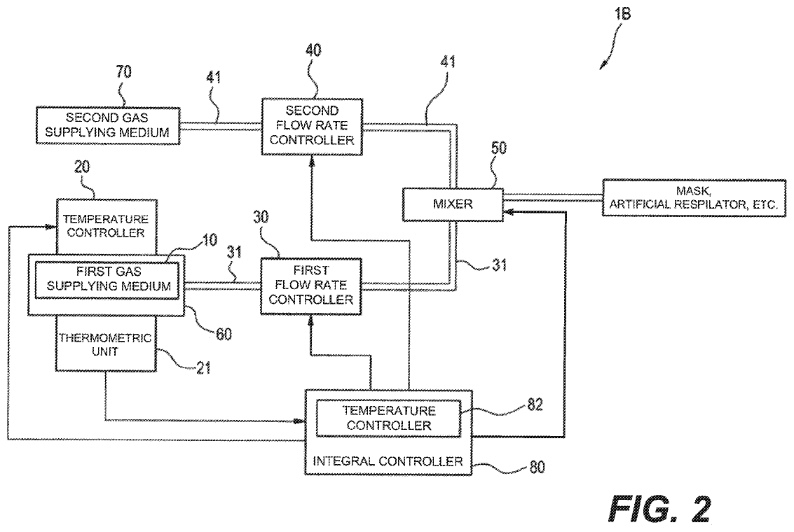

FIG. 2 is a functional block diagram illustrating a mixed gas supplying apparatus 1B according to a second embodiment. The second embodiment is obtained by adding a thermometric unit 21 and a temperature controller 82 to the configuration of the first embodiment. In FIG. 2, the other components that are the same as those in the first embodiment will be designated by the same reference numerals and repetitive explanations for those will be omitted.

In addition to the configuration of the mixed gas supplying apparatus 1A, the mixed gas supplying apparatus 1B comprises the thermometric unit 21 configured to measure the temperature associated with the first gas supplying medium 10 and the temperature controller 82 configured to control the temperature adjuster 20. The thermometric unit 21 is communicatively connected to the integral controller 80 and the temperature adjuster 20. The measurement results of the thermometric unit 21 are transmitted to the temperature controller 82 and stored therein.

The temperature measured by the thermometric unit 21 may be the temperature of the first gas inside the holder 60, the temperature of the first gas supplying medium 10 or the temperature of the holder 60. Furthermore, the timing of measurement by the thermometric unit 21 may be prior to the supply of the mixed gas from the mixed gas supplying apparatus 1B or may be after the initiation of the supply of the mixed gas from the mixed gas supplying apparatus 1B.

The temperature controller 82 is configured to control the temperature adjuster 20 depending on the temperature measured by the thermometric unit 21. The temperature controller 82 is communicatively connected to the temperature adjuster 20. The temperature controller 82 is configured such that various kinds of information associated with the temperature of the first gas supplying medium 10 have been stored therein. The various kinds of information associated with the temperature of the first gas supplying medium 10 may include, for example, the information on the generation of the first gas, such as graphs associated with the generated amount of hydrogen at the time when the temperature of the hydrogen storage alloy is changed, the target value of the concentration of the first gas in the mixed gas, and so on.

In this embodiment, the temperature controller 82 is configured to receive the value measured by the thermometric unit 21, to compare the measured value with the target value, to calculate the difference therebetween, and to change the setting of the temperature adjuster 20 so that the difference becomes zero.

The measurement of the temperature in the mixed gas supplying apparatus 1B, however, is not limited to the measurement of the temperature associated with the first gas supplying medium 10. For example, the mixed gas supplying apparatus 1B may be configured so that the thermometric unit 21 measures an ambient temperature, such as the atmospheric temperature in the space in which the holder 60 is disposed, and also configured so that the temperature controller 82 judges whether the room temperature is high or low, and controls the temperature adjuster 20, thereby controlling the heating of the holder 60.

Next, the operation of the mixed gas supplying apparatus 1B according to the second embodiment will be described with reference to FIG. 2.

When the power source (not shown) of the mixed gas supplying apparatus 1B is turned on, the thermometric unit 21 measures the temperature of the holder 60 as the temperature associated with the first gas supplying medium 10. The measurement result of the temperature of the holder 60 is transmitted from the thermometric unit 21 to the temperature controller 82. The temperature controller 82 reads the target temperature of the holder 60 having been registered in the temperature controller 82, compares the target temperature with the measured temperature, calculates the difference therebetween, and changes the setting of the temperature adjuster 20 so that the difference becomes zero, thereby adjusting the temperature of the holder 60, that is, the temperature associated with the first gas supplying medium 10.

Since the thermometric unit 21 and the temperature controller 82 are provided as described above, the temperature adjuster 20 can be controlled depending on the measurement result of the temperature, and the mixed gas having the concentration suitable for treatment can be obtained securely.

### Third Embodiment

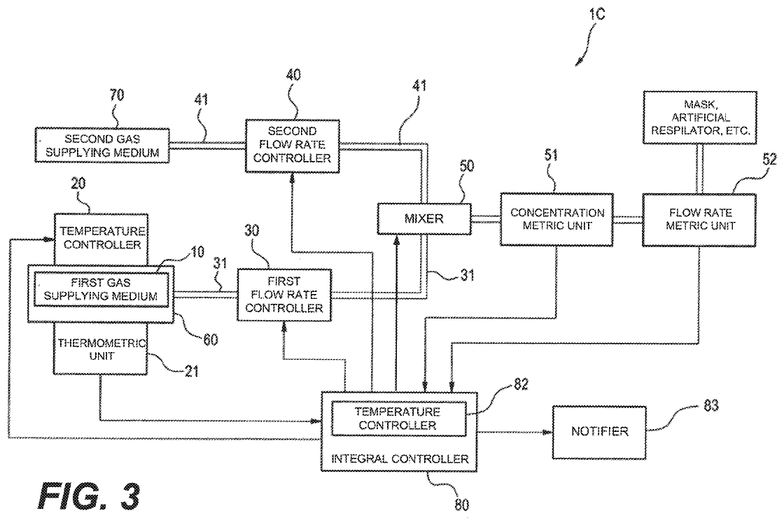

FIG. 3 is a functional block diagram illustrating a mixed gas supplying apparatus 1C according to a third embodiment. The third embodiment is obtained by adding a feedback control mechanism associated with the flow rate and/or concentration to the configuration of the first embodiment or the second embodiment. In FIG. 3, the other components that are the same as those in the second embodiment will be designated by the same reference numerals and repetitive explanations for those will be omitted.

The mixed gas supplying apparatus 1C comprises a concentration metric unit 51 and a flow rate metric unit 52 as the feedback control mechanism.

The concentration metric unit 51 is configured to measure the concentration of the first gas in the mixed gas that is prepared by the mixer 50. The concentration metric unit 51 is communicatively connected to the integral controller 80. The concentration measured by the concentration metric unit 51 is transmitted to the integral controller 80 and stored therein.

It should be noted that the concentration metric unit 51 can also be configured to measure the concentration of the second gas in the mixed gas additionally or alternatively to the concentration of the first gas in the mixed gas.

The flow rate metric unit 52 is configured to measure the flow rate of the mixed gas that is supplied from the mixer 50 to the outside of the mixed gas supplying apparatus 1C. The flow rate metric unit 52 is communicatively connected to the integral controller 80. The measurement result of the flow rate metric unit 52 is transmitted to the integral controller 80 and stored therein.

The integral controller 80 is configured to perform control to change the flow rate at the first flow rate controller 30 or the second flow rate controller 40 so that the concentration of the first gas in the mixed gas becomes the predetermined value on the basis of at least one of the received measurement results of the concentration and the flow rate.

In the case that at least one of the measurement results of the concentration and the flow rate has an abnormal value (for example, in the case that the value of the concentration is deviated from the desired value by a predetermined value or more), the integral controller 80 may stop the supply of the mixed gas. The stoppage of the supply of the mixed gas may be performed by stopping the supply of the mixed gas from the mixed gas supplying apparatus 1C to the outside and can be performed using various kinds of configurations and control methods. For example, the supply of the mixed gas may be stopped by the control performed by the integral controller 80 to stop the supply of the first gas from the first flow rate controller 30 and to stop the supply of the second gas from the second flow rate controller 40. Furthermore, it may be possible that an opening/closing valve (not shown) is provided between the mixer 50 and the mask, the artificial respirator or the like. This opening/closing valve is closed by the control of the integral controller 80 to stop the supply of the mixed gas from the mixed gas supplying apparatus 1C. Alternatively, in the case that at least one of the measurement results of the concentration and the flow rate has an abnormal value, the integral controller 80 may stop the supply of the first gas by controlling the first flow rate controller 30.

The mixed gas supplying apparatus 1C may include a notifier 83. The notifier 83 can be provided in various forms, for example, a compact electronic device, such as a tablet terminal or a smartphone, and a bedside monitor disposed on each bed in a hospital room.

The notifier 83 may display a warning or generate a warning sound associated with an abnormal operation of the mixed gas supplying apparatus 1C, may display a notice or generate a notice sound associated with a normal operation, or may display messages or generate message sounds associated with the others. As an example associated with an abnormal operation, it is possible to use a configuration in which, in the case that the operation for providing the mixed gas from the mixer 50 is performed even though the concentration of the first gas inside the mixer 50 has a high value of 3.5%, for example, an error message stating that “The concentration is too high” is displayed or an warning sound is generated from a buzzer, for example. As an example of the display or sound generation associated with a normal operation, it is possible to use a configuration in which the temperature associated with the first gas supplying medium 10 measured by the thermometric unit 21 is displayed or the target temperature having been stored in the integral controller 80 in advance is displayed. As an example of the display or sound generation associated with the others, it is possible to use a configuration in which texts describing the operation procedures of the mixed gas supplying apparatus 1C or the images illustrating the method for exchanging the holder 60 are displayed.

The mixed gas supplying apparatus 1C may be additionally provided with a filter (not shown). The filter can be provided at any position on the second supplying passage 41 (for example, between the second gas supplying medium 70 and the second flow rate controller 40) or on the supplying passage of the mixed gas (for example, between the mixer 50 and the mask, the artificial respirator or the like, between the flow rate metric unit 52 and the mask, the artificial respirator or the like).

Next, the operation of the mixed gas supplying apparatus 1C according to the third embodiment will be described with reference to FIG. 3.

After the power source (not shown) of the mixed gas supplying apparatus 1C is turned on and before the mixed gas is supplied from the mixer 50, the concentration metric unit 51 measures the concentration of the first gas in the mixed gas. Furthermore, after the power source (not shown) of the mixed gas supplying apparatus 1C is turned on and before the mixed gas is supplied from the mixer 50, the flow rate metric unit 52 measures the flow rate of the mixed gas. The concentration metric unit 51 transmits the measured concentration to the integral controller 80, and the flow rate metric unit 52 transmits the measured flow rate to the integral controller 80.

On the basis of the received information on the concentration and the flow rate, the integral controller 80 controls the flow rate of the first gas to be mixed in the first flow rate controller 30 and/or the flow rate of the second gas to be mixed in the second flow rate controller 40. For example, the integral controller 80 compares the concentration of the first gas in the mixed gas received from the concentration metric unit 51 with the target value of the concentration of the first gas in the mixed gas having been registered in the integral controller 80 in advance, calculates the difference therebetween, and changes the flow rate to be mixed in the first flow rate controller 30 so that the difference becomes zero. The control for making the difference to zero by the integral controller 80 may be performed by changing the flow rate of the second gas to be mixed in the second flow rate controller 40 or by changing the flow rate of the first gas to be mixed in the first flow rate controller 30 and the second flow rate controller 40.

Since the concentration metric unit 51 and the flow rate metric unit 52 are provided as described above, the first flow rate controller 30 or the second flow rate controller 40 is controlled so that the flow rate is changeable on the basis of the information on the flow rate of the mixed gas or the measured value of the concentration of the first gas in the mixed gas, whereby the mixed gas having the concentration suitable for treatment can be obtained securely. Moreover, since the supply of the mixed gas is stopped in the case that at least one of the measurement results of the concentration and the flow rate has an abnormal value, it is possible to prevent the mixed gas having an abnormally high concentration, for example, from being supplied to the patient. Furthermore, in the case that at least one of the measurement results of the concentration and the flow rate has an abnormal value, the first flow rate controller 30 stops the supply of the first gas, whereby it is possible to prevent the mixed gas having an abnormally high concentration, for example, from being supplied to the patient via a mask or the like.

### Fourth Embodiment

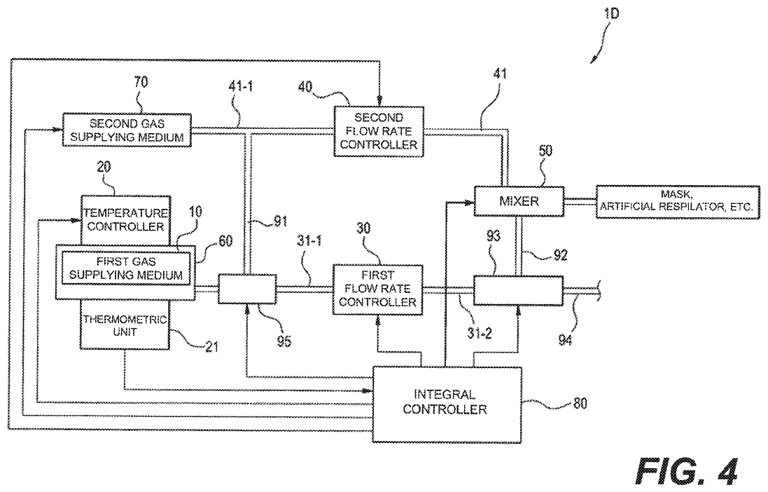

FIG. 4 is a functional block diagram illustrating a mixed gas supplying apparatus 1D according to a fourth embodiment. In FIG. 4, the other components that are the same as those in the second embodiment will be designated by the same reference numerals and repetitive explanations for those will be omitted.

Immediately after the supply of the mixed gas from the mixed gas supplying apparatus 1D is stopped, the first gas, the second gas and the mixed gas are in a state of remaining inside the mixed gas supplying apparatus 1D. Since the concentration of the mixed gas is herein adjusted to such a value suitable for inhalation and the second gas is medical air, no problem occurs even if mixed gas and the second gas remain inside the mixed gas supplying apparatus 1D.

However, the hydrogen gas serving as the first gas is flammable, and the state in which the first gas remains inside the mixed gas supplying apparatus 1D is not desirable. Hence, in this embodiment, the mechanism for discharging the first gas to the outside of the mixed gas supplying apparatus 1D is provided. The mixed gas supplying apparatus 1D comprises a first supplying passage for supplying the first gas from the first gas supplying medium 10 to the mixer 50 and a discharger for discharging the first gas remaining in the first supplying passage to the outside of the first supplying passage. In this embodiment, the supplying passage 31 in the first to third embodiments is replaced with the first supplying passage including a supplying passage 31-1 for connecting the first gas supplying medium 10 to the first flow rate controller 30 and a supplying passage 31-2 for connecting the first flow rate controller 30 to a first switching valve 93 (described later).

A second supplying passage 41-1 connects the second gas supplying medium 70 to the second flow rate controller 40. A discharged gas supplying passage 91 connects the supplying passage 31-1 to the second supplying passage 41-1. A second switching valve 95 is provided at the junction of the discharged gas supplying passage 91 and the supplying passage 31-1. The second switching valve 95 is preferably positioned close to the first gas supplying medium 10. In the case that the remaining first gas is desired to be discharged, the second switching valve 95 opens the passage between the supplying passage 31-1 and the discharged gas supplying passage 91 and closes the passage between the first gas supplying medium 10 and the supplying passage 31-1. Unless the remaining first gas is discharged, the second switching valve 95 closes the passage between the supplying passage 31-1 and the discharged gas supplying passage 91 and opens the passage between the first gas supplying medium 10 and the supplying passage 31-1.

The first flow rate controller 30 is connected to the first switching valve 93 via the supplying passage 31-1. The first switching valve 93 is connected to a supplying passage 92 that is connected to the mixer 50. Furthermore, the first switching valve 93 is connected to a discharging passage 94 communicating with the outside. The first switching valve 93 is switched to shut off or release the gas flow to the discharging passage 94 according to control signals from the integral controller 80. In the case of discharging the remaining first gas, the first switching valve 93 opens the passage to the discharging passage 94 (the outside) and closes the passage to the supplying passage 92. Furthermore, in the other cases (in the cases other than the case of discharging the remaining first gas), the first switching valve 93 closes the passage to the discharging passage 94 and opens the passage to the supplying passage 92.

Although the timing of discharging the remaining gas is not limited, the first gas remaining in the supplying passages 31-1 and 31-2 and the first flow rate controller 30 is discharged in some cases, for example, before the supply of the mixed gas from the mixed gas supplying apparatus 1D is started or after the supply of the mixed gas is finished. The flow of the processing in the case that the remaining first gas is discharged will be described below.

First, the integral controller 80 controls the first switching valve 93 so as to release the gas flow from the discharging passage 94. In other words, the first switching valve 93 opens the passage to the discharging passage 94 (the outside) for the second gas and the remaining first gas. The integral controller 80 then performs control so that the second gas is provided from the second gas supplying medium 70. The second switching valve 95 opens the passage between the discharged gas supplying passage 91 and the supplying passage 31-1. For example, when it is detected that the pressure of the second gas is low, the second switching valve 95 may be switched so as to open the passage between the discharged gas supplying passage 91 and the supplying passage 31-1. In other words, the integral controller 80 performs control so that the second gas flows into the supplying passages 31-1 and 31-2 and the first flow rate controller 30. By virtue of the above-mentioned control, the second gas is discharged to the outside via the discharged gas supplying passage 91, the supplying passages 31-1 and 31-2 (the first supplying passage), the first flow rate controller 30 and the discharging passage 94. At this time, the first gas remaining in the supplying passages 31-1 and 31-2 (the first supplying passage) and the first flow rate controller 30 is discharged to the outside together with the second gas.

After the time required for the discharge of the remaining first gas has elapsed, the integral controller 80 may shut off the gas flow from the first switching valve 93 to the discharging passage 94 (closes the passage of the gas to the outside) and deactivates the operation of the mixed gas supplying apparatus 1D. Additionally or alternatively, after the time required for the discharge of the remaining first gas has elapsed, the integral controller 80 may shut off the gas flow from the first switching valve 93 to the discharging passage 94 (closes the passage of the gas to the outside) and initiates the supply of the mixed gas in a manner similar to that of the first embodiment.

As described above, the discharged gas supplying passage 91 and the first switching valve 93 operate so as to serve as a discharger for discharging the first gas to the outside. More specifically, the discharged gas supplying passage 91 serves as a passage for supplying the second gas (the gas to be discharged) to the supplying passages 31-1 and 31-2 (the first supplying passage). When the remaining first gas is discharged, the first switching valve 93 opens so that the second gas having been supplied to the supplying passages 31-1 and 31-2 (the first supplying passage) is discharged to the outside. In other words, when the second gas and the remaining first gas are discharged, the first switching valve 93 opens the passage from the supplying passages 31-1 and 31-2 (the first supplying passage) to the discharging passage 94 (the outside).

The supplying passages 31-1 and 31-2 (the first supplying passage), the first flow rate controller 30, the first switching valve 93 and the supplying passage 92 illustrated in FIG. 4 constitute a passage for supplying the first gas from the first gas supplying medium 10 to the mixer 50. In this passage for supplying the first gas, the first gas remaining in the supplying passages 31-1 and 31-2 and the first flow rate controller 30 is pushed out to the outside by the discharging mechanism configured as described above. Hence, the first gas remaining inside the mixed gas supplying apparatus 1D is reduced, whereby the mixed gas supplying apparatus 1D can be maintained and stored more safely.

Although it has been described that the discharged gas supplying passage 91 is used to connect the supplying passage 31-1 to the second supplying passage 41-1, the discharged gas supplying passage 91 is not limited to be used for this connection. The discharged gas supplying passage 91 may be used to connect the supplying passage 31-1 to the supply source of the gas to be discharged (the second gas, such as medical air, oxygen, etc. in the above description) that serves to push out the first gas. In the configuration illustrated in FIG. 4, the discharged gas supplying passage 91 connects the supplying passage 31-1 to the second supplying passage 41-1 (the passage connecting the second gas supplying medium 70 to the second flow rate controller 40). With this configuration, the second gas supplying medium 70 can be used not only to serve as the supply source of the second gas constituting the mixed gas but also to discharge the first gas. Since the second gas supplying medium 70 can be used for a plurality of usages, the apparatus can be made simple and compact.

Furthermore, the first flow rate controller 30 can be configured to also have the function of the first switching valve 93. In other words, the first flow rate controller 30 may be configured to be able to perform both the flow rate control of the first gas and the control at the supply destination of the first gas. In this case, the first flow rate controller 30 is connected to the supplying passage 92 that is connected to the mixer 50. Furthermore, the first flow rate controller 30 is connected to the discharging passage 94 communicating with the outside. The control for releasing/shutting off the gas to/from the outside may be similar to the control performed by the above-mentioned first switching valve 93. In other words, the first flow rate controller 30 may have the function of the first switching valve 93 (the function for shutting off/releasing the gas flow from/to the outside.

### Fifth Embodiment

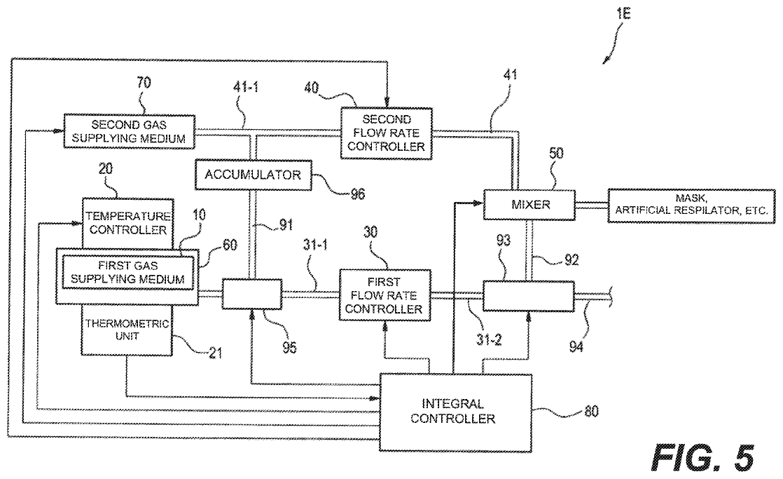

The discharger for the first gas is not necessarily limited to the configuration described with reference to the fourth embodiment. FIG. 5 is a functional block diagram illustrating a mixed gas supplying apparatus 1E according to a fifth embodiment. In FIG. 5, the other components that are the same as those in the fourth embodiment will be designated by the same reference numerals and repetitive explanations for those will be omitted. In this embodiment, an accumulator (ACC) is used as the discharger.

The mixed gas supplying apparatus 1E comprises an accumulator 96 disposed in the middle of the discharged gas supplying passage 91. The accumulator 96 is constructed so as to store the gas to be discharged (the second gas in this embodiment) in a cylinder or the like in an air tight manner and supplies the gas to be discharged to the supplying passage 31-1.

The flow of the processing in the case that the remaining first gas is discharged will be described below. The second gas from the second gas supplying medium 70 flows into the accumulator 96 at all times. As in the fourth embodiment, the integral controller 80 releases the gas flow from the first switching valve 93 to the outside when discharging the remaining first gas.

In the case that the pressure of the second gas supplied to the accumulator 96 has a predetermined value or more, the second gas is accumulated inside the accumulator 96. In the case that the pressure of the second gas supplied from the second gas supplying medium 70 is lowered to the predetermined value or less, the accumulator 96 supplies the second gas accumulated therein to the supplying passage 31-1. At this time, the second switching valve 95 opens the passage between the discharged gas supplying passage 91 and the supplying passage 31-1. The second switching valve 95 may operate so as to open the passage between the discharged gas supplying passage 91 and the supplying passage 31-1, for example, in the case that the pressure applied from the discharged gas supplying passage 91 is lowered to the predetermined value or less. The second gas is discharged to the outside via the supplying passage 31-1, the first flow rate controller 30, the supplying passage 31-2 and the discharging passage 94. The first gas remaining in the supplying passages 31-1 and 31-2 and the first flow rate controller 30 is also discharged to the outside together with the second gas.

After the time required for discharging the remaining first gas has elapsed, the integral controller 80 stops the discharging from the accumulator 96.

The first gas remaining in the supplying passages 31-1 and 31-2 (the first supplying passage) and the first flow rate controller 30 is also discharged by the above-mentioned configuration. This reduces a risk that the first gas remains inside the mixed gas supplying apparatus 1E, whereby the mixed gas supplying apparatus 1E can be maintained and stored more safely.

Although the accumulator 96 is provided on the discharged gas supplying passage 91 in the above-mentioned description, the accumulator 96 is not necessarily required to be provided at the position. The accumulator 96 may be provided at any position, provided that the accumulator 96 is provided on the upstream side of the passage connecting the supplying passages 31-1 and 31-2 and the first switching valve 93 and is configured to supply the gas to be discharged to the supplying passages 31-1 and 31-2 in the pushing-out manner.

The present disclosure is not limited to the configurations described with reference to the above embodiments, but can be modified or improved as necessary. In addition, the materials, shapes, forms, quantities, arrangement positions, etc. of the respective components in the above-mentioned embodiments may be arbitrary and not limited without departing from the concept of the present disclosure.

The present application is based on Japanese Patent Application No. 2017-006203 filed on Jan. 17, 2017, the entire contents of which are hereby incorporated by reference.

What is claimed is:

1. A mixed gas supplying apparatus comprising:
a temperature adjuster configured to adjust temperature associated with a first gas supplying medium for providing first gas to be mixed;
a first flow rate controller configured to control a flow rate of the first gas;
a second flow rate controller configured to control a flow rate of second gas to be mixed that is different from the first gas; and
a mixer configured to supply mixed gas in which the first gas supplied by the first flow rate controller and the second gas supplied by the second flow rate controller have been mixed.

2. The mixed gas supplying apparatus according to claim 1, comprising:
a thermometric unit configured to measure the temperature associated with the first gas supplying medium; and
a temperature controller configured to control the temperature adjuster in accordance with the temperature measured by the thermometric unit.

3. The mixed gas supplying apparatus according to claim 1, comprising:
a concentration metric unit configured to measure concentration of the first gas in the mixed gas.

4. The mixed gas supplying apparatus according to claim 3, comprising:
a flow rate metric unit configured to measure a flow rate of the mixed gas.

5. The mixed gas supplying apparatus according to claim 3, wherein:
the first flow rate controller is configured to change the flow rate of the first gas to be mixed in accordance with at least one of the flow rate and the concentration of the mixed gas.

6. The mixed gas supplying apparatus according to claim 3, wherein:
the second flow rate controller is configured to change the flow rate of the second gas to be mixed in accordance with at least one of the flow rate and the concentration of the mixed gas.

7. The mixed gas supplying apparatus according to claim 1, comprising:
a first supplying passage configured to supply the first gas from the first gas supplying medium to the mixer; and
a discharger configured to discharge the first gas remaining in the first supplying passage to an outside of the first supplying passage.

8. The mixed gas supplying apparatus according to claim 7, wherein: the discharger comprises a switching valve configured to open a passage connecting the first supplying passage to the outside when the first gas remaining in the first supplying passage is discharged.

9. The mixed gas supplying apparatus according to claim 8, comprising:
a second supplying passage configured to supply the second gas from a second gas supplying medium to the second flow rate controller, wherein:
the discharger comprises a discharged gas supplying passage connecting the first supplying passage and the second supplying passage.

10. The mixed gas supplying apparatus according to claim 9, wherein:
the discharged gas supplying passage is provided with an accumulator configured to cause the second gas to flow from the second supplying passage to the switching valve.

11. The mixed gas supplying apparatus according to claim 1, comprising:
an integral controller configured to control the first flow rate controller and the second flow rate controller such that provision of the first gas is initiated after provision of the second gas is initiated.

12. The mixed gas supplying apparatus according to claim 1, comprising:
a concentration metric unit configured to measure concentration of the first gas in the mixed gas; and
a flow rate metric unit configured to measure a flow rate of the mixed gas, wherein:
supply of the mixed gas from the mixer is stopped in a case that at least one of the concentration measured by the concentration metric unit and the flow rate measured by the flow rate metric unit exhibits an abnormal value.

13. The mixed gas supplying apparatus according to claim 1, comprising:
a concentration metric unit configured to measure concentration of the first gas in the mixed gas; and
a flow rate metric unit configured to measure a flow rate of the mixed gas, wherein:
supply of the first gas from the first flow rate controller is stopped in a case that at least one of the concentration measured by the concentration metric unit and the flow rate measured by the flow rate metric unit exhibits an abnormal value.

14. A method of supplying mixed gas comprising steps of:
measuring temperature associated with a first gas supplying medium;
adjusting the temperature in accordance with a comparison result between a measured value of the temperature and a target value of the temperature;
controlling a flow rate of first gas to be mixed so as to fall within a predetermined range;
controlling a flow rate of second gas to be mixed that is different from the first gas so as to fall within a predetermined range;
mixing the first gas and the second gas to prepare mixed gas; and
supplying the mixed gas.
